(19) United States
(12) Patent Application Publication
(10) Pub. No.: US 2007/0219172 A1
(43) Pub. Date: Sep. 20, 2007

(54) Selective testicular 11beta-HSD inhibitors for the treatment of hypergonadism associated disorders and modulation of fertility

(75) Inventors: David J. Morris, Barrington, RI (US); Syed Abdul Latif, Pawtucket, RI (US); Matthew P. Hardy, New York, NY (US); Renshan Ge, Albany, NY (US)

Correspondence Address: LAHIVE & COCKFIELD, LLP, ONE POST OFFICE SQUARE, BOSTON, MA, 02109-2127, US

(73) Assignees: The Miriam Hospital, Providence, RI (US); The Population Council, New York, NY (US)

(21) Appl. No.: 11/595,826

(22) Filed: Nov. 10, 2006

Related U.S. Application Data
(60) Provisional application No. 60/735,451, filed on Nov. 10, 2005.

(51) Int. Cl. A61K 31/573; A61K 31/57

(52) U.S. Cl. 514/177; 514/179

(57) ABSTRACT

Methods for modulating testosterone levels using selective 11β-HSD1-dehydrogenase, 11β-HSD1-reductase and 11β-HSD2 dehydrogenase modulating compounds are described.

### RELATED APPLICATIONS

This application claims priority to U.S. Provisional Patent Application Ser. No. 60/735,451, filed on Nov. 10, 2005, the entire contents of which are hereby incorporated herein by reference.

### BACKGROUND

Corticosteroids, also referred to as glucocorticoids are steroid hormones, the most common form of which is cortisol. Modulation of glucocorticoid activity is important in regulating physiological processes in a wide range of tissues and organs. High levels of glucocorticoids may result in excessive salt and water retention by the kidneys, producing high blood pressure.

Glucocorticoids (GC's) play an important role in the regulation of vascular tone and blood pressure. Glucocorticoids can bind to and activate the glucocorticoid receptor (GR) and, possibly, the mineralocorticoid receptor (MR)) to potentiate the vasoconstrictive effects of both catecholamines and angiotensin II (Ang II). Tissue glucocorticoid levels are regulated by two isoforms of the enzyme 11β-hydroxysteroid dehydrogenase (11β-HSD). 11β-HSD converts glucocorticoids into metabolites that are unable to bind to MRs (Edwards C R et al. (1988) Lancet 2:986-9; Funder et al., (1988) Science 242, 583, 585).

### SUMMARY OF THE INVENTION

In one embodiment, the invention pertains to a method for increasing male fertility. The method includes administering an effective amount of a 11β-HSD1 reductase inhibitor to a subject, such that said fertility is increased, wherein the 11β-HSD1 reductase inhibitor is a non-competitive inhibitor. In another embodiment, the 11β-HSD1 reductase inhibitor is a competitive inhibitor selected from: 3β,5α-reduced steroids, 3α,5α-TH-cortisone, 3α,5β-TH-cortisone, 5α-DH-corticosterone, 3α,5α-TH-corticosterone, 3α,5α-TH-11-dehydro-corticosterone, 11β-OH-pregnanolone, 11-keto-allopregnanolone, 11-keto-androstenedione, and pharmaceutically acceptable prodrug or salts thereof.

In another embodiment, the invention pertains, at least in part, to a method for increasing testosterone levels in a subject. The method includes administering to the subject an effective amount of a 11β-HSD1 reductase inhibitor, such that testosterone levels in the subject are increased, wherein the 11β-HSD1 reductase inhibitor is a non-competitive inhibitor. In another embodiment, the 11β-HSD1 reductase inhibitor is a competitive inhibitor selected from: 3β,5α-reduced steroids, 3α,5α-TH-cortisone, 3α,5β-TH-cortisone, 5α-DH-corticosterone, 3α,5α-TH-corticosterone, 3α,5α-TH-11-dehydro-corticosterone, 11β-OH-pregnanolone, 11-keto-allopregnanolone, 11-keto-androstenedione, and pharmaceutically acceptable prodrug or salts thereof.

In another embodiment, the invention pertains at least in part, to a method for decreasing male fertility. The method includes administering to a subject an effective amount of a 11β-HSD1 dehydrogenase inhibitor, such that fertility is decreased. In one embodiment, the inhibitor is a non-competitive inhibitor. In another embodiment, the inhibitor is a 3β,5α-reduced steroid, 3α,5α-TH-aldosterone, 3α,5α-TH-cortisol, 5α-DH-corticosterone, 3α,5α-TH-corticosterone, 3α,5α-TH-11-dehydro-corticosterone-11β-OH-allopregnanolone, 11β-OH-pregnanolone, 11β-OH-androstanediol, or a pharmaceutically acceptable prodrug or salt thereof.

In yet another embodiment, the invention pertains at least in part to a method for decreasing testosterone levels in a subject. The method includes administering to a subject an effective amount of a 11β-HSD1 dehydrogenase inhibitor, such that testosterone levels in the subject are decreased. In a further embodiment, the inhibitor is non-competitive. In another embodiment, the inhibitor is a 3β,5α-reduced steroid, 3α;5α-TH-aldosterone, 3α,5α-TH-cortisol, 5α-DH-corticosterone, 3α,5α-TH-corticosterone, 3α,5α-TH-11-dehydro-corticosterone, 11β-OH-allopregnanolone, 11β-OH-pregnanolone, 11β-OH-androstanediol, or a pharmaceutically acceptable prodrug or salt thereof.

In another embodiment, the invention pertains to a method of increasing testosterone biosynthesis. The method includes administering to a male subject an effective amount of a 11β-OH steroid or 11-keto steroid, such that testosterone biosynthesis is increased.

In yet another embodiment, the invention also pertains, at least in part, to a method of decreasing testosterone biosyntheis. The method includes administering to a male subject an effective amount of an 1-deoxy steroid, such that testosterone levels are decreased.

In yet another embodiment, the invention also pertains, at least in part, to a method of treating a hypergonadism associated disorder in a subject, e.g., a male subject. The method includes administering to a subject an effective amount of a 11β-HSD1 reductase inhibitor, such that the hypergonadism associated disorder is treated.

### DETAILED DESCRIPTION OF THE INVENTION

I. Glucocorticoids and 11β-HSD1 Reductase, 11β-HSD1 Dehydrogenase and 11β-HSD2 Dehydrogenase

Glucocorticoids can affect vascular tone by modifying the actions of several vasoactive substances. Glucocorticoids amplify the vasoconstrictive actions of a-adrenergic catecholamines and angiotensin II on vascular smooth muscle cells. It has been reported that glucocorticoids decrease the biosynthesis of both nitric oxide and prostaglandin I, and attenuate the vasorelaxant actions of atrial natriuretic peptide in vascular tissue. Thus, the multiple effects of glucocorticoids in vascular tissue operate to increase vascular tone. Since vascular smooth muscle cells contain both glucocorticoid (GR) and mineralocorticoid (MR) receptors it is possible that glucocorticoids could mediate their effects in vascular tissue via either or both of these receptor types.

Glucocorticoids (GC's) are metabolized in vascular and other tissue by two isoforms of 11β-hydroxysteroid dehydrogenase (11β-HSD1). 11β-HSD2 is unidirectional and metabolizes glucocorticoids to their respective inactive 11-dehydro derivatives. 11β-HSD1 is bi-directional, also possessing reductase activity and thus the ability to regenerate active glucocorticoids from the 11-dehydro derivatives. In vascular tissue, glucocorticoids amplify the pressor responses to catecholamines and angiotensin II and may down-regulate certain depressor systems such as nitric oxide and prostaglandins. Both 11β-HSD2 and 11β-HSD1 are believed to regulate glucocorticoid levels in vascular tissue and are part of additional mechanisms that control vascular tone.

Glucocorticoids are known to play an important role in the regulation of vascular tone and blood pressure. Glucocorticoid receptors (GR) and mineralocorticoid receptors (MR) are present in aorta, mesenteric arteries and VSM cells in culture. Glucocorticoids can bind to and activate GR (and possibly MR) to potentiate the vasoconstrictive effects of both catecholamines and Ang II. Human and rat vascular endothelial cells contain both 11β-HSD2 and 11β-HSD1, 11β-HSD2 uses NAD⁺ as a co-factor and acts only as a dehydrogenase converting glucocorticoids to their inactive 11-dehydro metabolites. It is generally understood that 11β-HSD2 operates to protect both MR and GR from excessive stimulation by glucocorticoids and we and others have shown that glucocorticoids further amplify the contractile effects of phenylephrine and Ang II when 11β-HSD enzyme activity is inhibited.

11β-HSD1 uses NADP⁺ as a co-factor and is bi-directional functioning as both a reductase and dehydrogenase. Using RT-PCR, it has been shown that rat vascular smooth muscle (VSM) cells only contain 11β-HSD1, which under “physiologic conditions” acts largely as a reductase (3 reductase to 1 dehydrogenase) generating active corticosterone from inactive 11-dehydro-corticosterone.

11β-HSD1 reductase has an important role as a generator of active GC in vascular tissue. 11β-HSD1 inactivates glucocorticoid molecules, allowing lower circulating levels of aldosterone to maintain renal homeostasis. Human and rat vascular endolethial cells (EC) contain both 11β-HSD1 and 11β-HSD2. 11β-HSD2 uses NAD+ as a co-factor and acts only as a dehydrogenase converting glucocorticoids to their inactive 11-dehydro metabolites.

11β-HSD2 operates to protect both MR and GR from excessive stimulation by glucocorticoids and it has been shown that glucocorticoids further amplify the contractile effects of phenylephrine (PE) and Ang II when 11β-HSD1 or 2 dehydrogenase enzyme activity is inhibited.

Working with freshly prepared rat Leydig cells, it was shown that certain 11β-hydroxylated and 11-keto derivatives of androgens and progestogens are potent selective inhibitors of 11β-HSD1 (11β-hydroxy steroid dehydrogenase), an enzyme present in testicular Leydig cells. These cells have been shown to regulate the effects of glucocorticoids on testosterone biosynthesis.

These substances may either inhibit the inactivation of active glucocorticoids by 11β-HSD1 dehydrogenase or inhibit the regeneration of active glucocortcoids by 11β-HSD1 reductase. It has been shown that the testis are able to synthesize several of these substances and that inhibitors may also be locally synthesized.

Inhibitors which cause testicular levels of corticosterone in rodents or cortisol in humans to increase would decrease production of testosterone, whereas those which cause them to decrease would increase testosterone production.

II. Methods of Modulating Male Fertility

Lower concentrations of glucocorticoids stimulate spermatogenesis. Therefore, 11β-HSD1 reductase inhibitors may be used to treat infertility. In contrast, 11β-HSD1 dehydrogenase inhibitors and 11β-HSD2 dehydrogenase inhibitors may be used decrease fertility.

The invention includes a method for increasing male fertility. The method includes administering an effective amount of a selective 11β-HSD1 reductase inhibitor to a subject, such that fertility is increased. The invention also includes a method for increasing testosterone levels by administering to a subject an effective amount of a 11β-HSD1 reductase inhibitor.

In another embodiment, the invention features a method for decreasing male fertility. The method includes administering an effective amount of a selective 11β-HSD1 dehydrogenase and/or a selective 11β-HSD2 dehydrogenase inhibitor. The invention also includes a method for decreasing testosterone levels in a subject by administering to said subjects an effective amount of a selective 11β-HSD1 dehydrogenase and/or a selective 11β-HSD2 dehydrogenase inhibitor, such that testosterone levels are decreased in said subject.

The term “subject” includes subjects which modulation of testosterone levels is desired, such as mammals. Examples of mammals include dogs, cats, bears, rabbits, mice, rats, goats, cows, sheep, horses, and, preferably, humans. The subject may be suffering from or at risk of suffering from infertility or a hypergonadism associated disorder. In a further embodiment, the subject is male.

The term “effective amount” of the 11β-HSD1 reductase, 11β-HSD1 dehydrogenase, or 11β-HSD2 dehydrogenase modulating compound is that amount necessary or sufficient to modulate testosterone levels in a subject so that a desired effect, e.g., increasing or decreasing fertility or treating a hypergonadism associated disorder, is obtained. The effective amount can vary depending on such factors as the size and weight of the subject, or the particular 11β-HSD1 reductase, 11β-HSD1 dehydrogenase, or 11β-HSD2 dehydrogenase modulating compound, e.g., inhibiting, compound.

In a further embodiment, the 11β-HSD1 reductase, 11β-HSD1 dehydrogenase, or 11β-HSD2 dehydrogenase modulating compound may be administered in combination with a pharmaceutically acceptable carrier.

The language “in combination with” another agent includes co-administration of the compound of the invention and the agent, administration of the compound of the invention first, followed by the other agent and administration of the other agent first, followed by the compound of the invention.

III. Methods of Treating Hypergonadism Associated Disorders and Regulation of Testosterone and NADPH Production

It has been found that testicular production of testosterone is directly dependant on the adrenal corticosteroid hormones, cortisol and corticosterone, and other specific 11β-OH-steroid metabolites derived from the adrenal gland. This finding is unexpected and implies that normal levels of glucocorticoids stimulate testosterone in contrast to the suppressive effects of excessive glucocorticoid exposures characteristic of stress. See for example, Abstract P2-318, Endocrine Society Meeting, June 2005; and Latif S A, et al. 2005 Endogenous selective inhibitors of 11β-hydroxysteroid dehydrogenase isoforms 1 and 2 of adrenal origin. Mol Cell Endocrinol.

In particular, endogenous, adrenally derived steroids can regulate the set point/functional equilibrium of 11β-HSD1 dehydrogenase and 11β-HSD1 reductase (2). It has also been found that locally synthesized potent enzyme 11β-OH-inhibitors may also play a regulatory role by acutely stimulating testosterone production by a rapid non-genomic mechanism which supplies NADPH for testosterone biosynthesis. Compounds which stimulate testosterone production include those that possess an 11β-OH group, (such as 11β-OH-testosterone produced by testicular CYP11B1), 17β-HSD1 reduction of adrenally synthesized 11β-OH-androstenedione, and 3α,5α- or 3β,5α-ring A-reduced derivatives. Locally synthesized potent enzyme inhibitors possessing the 11-keto group such as 11-keto-testosterone and its 3α,5α- or 3β,5α-ring A-reduced derivatives also feed NADP⁺ into this NADPH regenerating system and, likewise, acutely stimulate testosterone production. Thus, both adrenally and locally synthesized endogenous inhibitors have this additional role depending on the threshold of circulating and stress levels of cortisol and corticosterone required for suppression of testosterone biosynthesis.

One of the rate-limiting factors in testosterone biosynthesis is the availability of co-factor NADPH. Until recently, it was thought that a major source of NADPH in testis was glucose-6-phosphate dehydrogenase. Recently, using whole cell preparations of rat Leydig cells, it has been shown that certain competitive substrate inhibitors of 11β-HSD1 dehydrogenase can, like corticosterone, serve to regenerate NADPH and stimulate in a coupled, cooperative manner testosterone biosynthesis. Just as hexose-6-phosphate dehydrogenase is coupled to the 11β-HSD1 reductase activity in liver, testicular 11β-HSD1 similarly is coupled to the steroidogenic enzymes for the synthesis of testosterone. The very high levels of 11β-HSD1 present in testicular Leydig cells thus, plays more than one role; with one being to prevent high levels of glucocorticoid (e.g. acquired during stress) resulting in lower testosterone production (through a genomic effect exerted via GR receptor pathway) and another to provide the main source for the regeneration of NADPH via coupling of 11β-HSD1 dehydrogenase with the steroidogenic enzymes during testosterone biosynthesis (the non-genomic effect).

In one embodiment, the invention pertains to a method of reducing visceral fat in a subject, comprising administering an effective amount of an 11β-OH steroid, such that the visceral fat in said subject is reduced. In a further embodiment, the subject is male. In other embodiments, the invention pertains to methods for treating obesity, insulin resistance, metabolic syndrome and decreasing abdominal fat mass, by administering to a subject an effective amount of an 11β-steroid or other 11β-HSD inhibitor.

One prevailing symptom of obesity, insulin resistance, and the metabolic syndrome is due to hypogonadism in men. Intervention studies have demonstrated that correction of relative hypogonadism, through increased testosterone levels, in men with visceral obesity and other manifestations of the metabolic syndrome, decreases abdominal fat mass and reverses glucose intolerance, as well as lipoprotein abnormalities in the serum. Further analysis of the underlying mechanism has also disclosed a regulatory role for testosterone in counteracting visceral fat accumulation. Thus, the competitive substrate inhibitors, the 11β-OH-steroids listed above can be used to increase testosterone levels and thereby reduce risk of onset for these other diseases.

This is in contrast to glucocorticoid overexposure, which has profoundly suppressive effects on Leydig cell function, greatly lowering testosterone production. In acute and chronic stresses, local elevation of testicular glucocorticoid levels are highly correlated with lower testosterone levels. However, the 11β-OH-steroids listed above, like non-stress levels of cortisol and corticosterone, can be used to increase testosterone levels in hypogonadism and also to improve fertility.

In addition, testicular Leydig cell 11β-HSD1 is an NADPH generating system, in which adrenal glucocorticoids serve as the main source to drive the enzymatic reaction. In the testis, this system is the equivalent of, and substitutes for, hexose 6-phosphate dehydrogenase (H6PDH) which is one of the suppliers of NADPH in the liver and many other target tissues of glucocorticoids. Furthermore, 11β-HSD1 operates in co-operative, coupled reactions, together with steroidogenic enzymes leading to synthesis of testosterone and serves as an NADPH regenerating system, and therefore regulates testosterone levels.

In one embodiment, the invention pertains to a method of increasing testosterone biosynthesis by administering a competitive substrated such as an 11β-OH-steroid. The 11-β-OH steroid drives the generation of NADPH and feeds the NADPH regeneration system(s) leading to an increase in testosterone biosynthesis and circulating testosterone levels. However, it should be noted that as a consequence of 11β-HSD1-dehydrogenase inhibition by dehydrogenase inhibitors (at a threshold level or during stress when cortisol or corticosterone is present in large quantities), cortisol and/or corticosterone accumulate and bind to the GR, thereby genomically suppressing testosterone production.

In another embodiment, the invention pertains to a method for increasing the rate of regeneration of NADPH, by administering an effective amount of a 11-keto steroid (such as, but not limited to, cortisone and 11-dehydrocorticosterone). It has been found that 11-keto-steroids which act as competitive substrates drive the regeneration of NADP⁺ (via the reductase) and likewise feed and speed up the NADPH regeneration system(s) and testosterone biosynthesis. NADP⁺ generally should not be rate-limiting because, in addition to the 11βHSD1 reductase, 2 to 3 NADP⁺ molecules are generated for each molecule of testosterone synthesized. Conversely, cortisone, and/or 11-dehydro-corticosterone, will accumulate, as a consequence of 11β-HSD1 reductase inhibition, lowering cortisol and corticosterone levels, and that will result in de-inhibition of genomically suppressed testosterone production.

It has also been found that 11-deoxy steroids, such as 3α,5β-terahydro-deoxycorticosterone and 3α,5β-tetrahydro-progesterone and chenodeoxycholic acid (potent inhibitors of 11β-HSD1 dehydrogenase), do not generally participate as substrates in 11β-HSD reactions. Thus, they stop generation of NADPH and therefore, testosterone biosynthesis and also, will cause cortisol and corticosterone to accumulate and bind GRs, genomically suppressing testosterone production. These inhibitors can be used for decreasing fertility and for the treatment of prostate disease and prostate cancer.

Similarly, non-competitive 11β-OH inhibitors (such as chemically synthesized steroid derivatives described herein) will not participate in these reactions; thus will stop generation of NADPH, and will cause cortisol and corticosterone to accumulate and bind GR and genomically suppress testosterone production. Secondly, they also decrease rates of testosterone biosynthesis. These inhibitors can be used for decreasing fertility purposes and for the treatment of prostate disease and prostate cancer.

Non-competitive 11-keto inhibitors (such as chemically synthesized steroid derivatives described herein) will not participate in this reaction. Therefore, they will stop generation of NADP⁺ because of 11β-HSD1 reductase inhibition. This will cause cortisone and 11-dehydro-corticostrone to accumulate, de-inhibiting the genomic suppression and increasing testosterone production. Secondly, they will also increase the rate of testosterone biosynthesis due to inhibition of NADPH utilization by 11β-HSD1 reductase. These inhibitors can be used for treatment of hypogonadism associated states by increasing testosterone levels. These compounds also may increase fertility. Similarly, these compounds may also be used for the treatment of hypergonadism associated disorders, such as obesity, insulin resistance and metabolic syndrome.

In one embodiment, the invention also pertains to a method for treating hypergonadism associated disorders, by administering to a subject an effective amount of an 11β-HSD inhibitor. In a further embodiment, the 11β-HSD inhibitor is a non-competitive inhibitor, e.g., a steroid derivative.

The term “hypergonadism associated disorders” include disorders which can be treated by increasing testosterone levels in a subject. These disorders include obesity, metabolic syndrome, insulin resistance and diabetes.

IV. 11β-HSD1 Reductase Modulating Compounds, 11β-HSD1-Dehydrogenase Modulating Compounds and 11β-HSD2 Dehydrogenase Modulating Compounds

The term “11β-HSD1 reductase modulating compound” include compounds and agents (e.g., oligomers, proteins, etc.) which modulate or inhibit the activity of 11β-HSD1 reductase. In an advantageous embodiment, the 11β-HSD1 reductase modulating compound is an 11β-HSD1 reductase inhibitor (also referred to as “11β-HSD1 reductase inhibiting compound”). The 11β-HSD1 reductase modulating compound may be a small molecule, e.g., a compound with a molecular weight below 10,000 daltons.

In a further embodiment, the 11β-HSD1 reductase modulating compound is a selective inhibitor of 11β-HSD1 reductase. The term “selective 11β-HSD1 reductase inhibitor” includes compounds which selectively inhibit the reductase activity of 11β-HSD1 as compared to the dehydrogenase activity. In a further embodiment, the reductase activity is inhibited at a rate about 2 times or greater, about 3 times or greater, about 4 times or greater, about 5 times or greater, about 10 times or greater, about 15 times or greater, about 20 times or greater, about 25 times or greater, about 50 times or greater, about 75 times or greater, about 100 times or greater, about 150 times or greater, about 200 times or greater, about 300 times or greater, about 400 times or greater, about 500 times or greater, about 1×10³ times or greater, about 1×10⁴ times or greater, about 1×10⁵ times or greater, or about 1×10⁶ or greater as compared with the inhibition of the dehydrogenase activity of 11β-HSD1.

In a further embodiment, the 11β-HSD1 reductase modulating compound may be a steroid or a steroid derivative. The steroid ring system is generally numbered according to IUPAC conventions, as shown below:

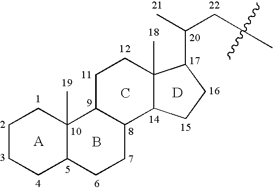

Examples of 11β-HSD1 reductase modulating compounds include 11-keto steroid compounds, e.g., compounds with the steroid ring system with a carbonyl functional group at the 11-position of the steroid ring. Examples of steroid compounds with an 11-keto group include, for example, 11-keto progesterone, 11-keto-testosterone, 1-keto-androsterone, 1-keto-pregnenolone, 11-keto-dehydro-epiandrostenedione, 3α, 5α-reduced-11-ketoprogesterone, 3α,5α-reduced-11-keto-testosterone, 3α,5α-reduced-11-keto-androstenedione, 3α,5α-tetrahydro-11-dehydro-corticosterone, 3α,5α-reduced-11-keto-pregnenolone, and 3α,5α-reduced-11-keto-dehydro-epiandrostenedione. Other examples of 11β-HSD1 reductase modulating compounds of the invention are compounds which conserve a least a portion of the steroid nucleus. These compounds may have additional substituents, such as fatty acid tails at the 22 position, or other modifications (e.g., substitutions of the ring by halogens, formation of esters or other protecting groups for the hydroxyl groups of the steroids, or replacement of functional groups with others that may, for example, advantageously, lengthen the time the molecule is in its active form in a subjects body. Alternatively, the modifications can be such that the reduce the time the compound is in its active form in a subject's body.

Other examples of 11β-HSD1 reductase inhibiting compounds include 3α,5α-TH-cortisone, 3α,5β-TH-cortisone, 5α-DH-corticosterone, 3α,5α-TH-corticosterone, 3α,5α-TH-11-dehydro-corticosterone, 11β-OH-pregnanolone, 11-keto-allopregnanolone, and 11-keto-androstenedione.

Examples of 11β-HSD1 reductase modulating compounds also include 3α,5α-reduced steroid compounds. Examples of 3β,5α-reduced steroid compounds include 11-keto-3β,5α-TH-testosterone. Examples of 3α,5α-reduced steroid compounds include, 3α,5α-reduced-11-ketoprogesterone, 3α,5α-reduced-11-keto-testosterone, 3α, 5α-reduced-11-keto-androstenedione, 3α,5α-tetrahydro-11-dehydro-corticosterone, 3α, 5α-reduced-11-keto-pregnenolone, 3α, 5α-reduced corticosterone, 3α,5α-reduced progesterone, 3α,5α-reduced testosterone and 3α,5α-reduced-11-keto-dehydro-epiandrostenedione.

Examples of 11β-HSD1 reductase inhibiting compounds also include 3β, 5α-reduced steroids. These compounds may also include a keto group at the 11 position. The term “3β,5α reduced steroids” includes compounds with a steroid ring structure and a 3β,5α conformation at the 3 and 5 positions, as described above. These compounds may be further substituted with other substituents known in the art. Examples of 3α,5β reduced steroids include 11-keto-3β,5α-TH-testosterone, 3β,5α-reduced-11-ketoprogesterone, 3β,5α reduced-11-keto-androstenedione, 3β,5α-tetrahydro-11-dehydro-corticosterone, 3β, 5α-reduced-11-keto-pregnenolone, 3β, 5α-reduced-11-keto-dehydro-epiandrostenedione, 3β,5α-reduced deoxycorticosterone, 3β,5α-reduced progesterone, 3β,5α-reduced testosterone, and pharmaceutically acceptable salts and prodrugs thereof.

In a further embodiment, the 11β-HSD1 reductase modulating compound is 3α, 5β reduced, e.g., 3α,5β-reduced deoxycorticosterone.

The invention also pertains to derivatives of the compounds described herein, such as steroid derivatives with a steroid ring structure optionally substituted with additional substituents which allow the compound to perform its intended function. Examples of such modifications include compounds modified with acetylenic groups (e.g., 17-acetylenic steroids), alkyl groups (e.g., 2α-alkyl (e.g., methyl), 12α-alkyl, 12β alkyl), halogenation, (e.g., 9α-halogenated, e.g., 9α-fluorinated, etc.), esters (e.g., succinates, hemi-succinates, carbohydrates, glucoronides, glutarates, etc.), or additional unsaturations (e.g., Δ1,2-unsaturated steroids). It should be noted that the steroid compounds may be converted to the active form of the modulating compound within the subject. The invention includes administering compounds which are in other forms, e.g., prodrugs, and which are metabolized in vivo to yield the compounds described herein. Additional modifications can be found in Human Adrenal Cortex, Ciba Symposium, edited by Perry Symington, 1962. In one embodiment, the steroid derivatives are non-competitive inhibitors.

In another embodiment, the 11β-hydroxylated progesterone compounds are protected such that 20α- or 20β-HSD enzymatic reduction of the side-chain is reduced or prevented. In another embodiment, the 11β-hydroxylated testosterone compounds are protected such that 17β-HSD enzymatic oxidation of the 17β-hydroxyl grouping is slowed or prevented.

In one embodiment, the 11β-HSD1 reductase inhibitors possess IC₅₀'s less than about 0.5 μM using 600 nanoM 11-dehydro-corticosterone substrate concentration and testicular leydig cell homogenates. Methods for testing the IC₅₀'s of the enzymes are described in further detail in Latif, S. A. et al. Steroids 62: 230-237, 1997. In another embodiment, the 11β-HSD1 reductase inhibitors have an IC₅₀ of 80 μM or less, or, preferably, 15 μM or less. In another embodiment, the 11β-HSD1 reductase inhibitors have an IC₅₀ of less than 100 μM.

Other examples of 11β-HSD1 reductase modulating compounds include carbenoxolone and derivatives thereof.

The term “11β-HSD1 dehydrogenase modulating compound” include compounds and agents (e.g., oligomers, proteins, etc.) which modulate or inhibit the activity of 11β-HSD1 dehydrogenase. In an advantageous embodiment, the 11β-HSD1 dehydrogenase modulating compound is an 11β-HSD1 dehydrogenase inhibitor (also referred to as “11β-HSD1 dehydrogenase inhibiting compound”). The 11β-HSD1 dehydrogenase modulating compound may be a small molecule, e.g., a compound with a molecular weight below 10,000 daltons.

In a further embodiment, the 11β-HSD1 dehydrogenase modulating compound is a selective inhibitor of 11β-HSD1 dehydrogenase. The term “selective 11β-HSD1 dehydrogenase inhibitor” includes compounds which selectively inhibit the dehydrogenase activity of 11β-HSD1 as compared to the reductase activity of 11β-HSD1. In a further embodiment, the dehydrogenase activity is inhibited at a rate about 2 times or greater, about 3 times or greater, about 4 times or greater, about 5 times or greater, about 10 times or greater, about 15 times or greater, about 20 times or greater, about 25 times or greater, about 50 times or greater, about 75 times or greater, about 100 times or greater, about 150 times or greater, about 200 times or greater, about 300 times or greater, about 400 times or greater, about 500 times or greater, about 1×10³ times or greater, about 1×10⁴ times or greater, about 1×10⁵ times or greater, or about 1×10⁶ or greater as compared with the inhibition of the reductase activity of 11β-HSD1.

In one embodiment, the 11β-HSD1 dehydrogenase inhibitor is a small molecule, such as a steroid or a derivative thereof. In a further embodiment, the steroid is 3α,5β-reduced. Examples of 3α,5β-reduced steroids include 3α,5β-reduced-11β-OH-progesterone, 3α,5β-reduced-11β-OH-testosterone, chenodeoxycholic acid, 3α,5β-reduced-pregnenolone, 3α,5β-reduced-dehydro-epiandrostenedione, 3α,5β-reduced-progesterone, 3α,5α-reduced deoxycorticosterone, 3α,5β-reduced-chenodeoxycholic acid, 3α,5β-reduced progesterone, 3α,5β-reduced testosterone, 3α,5β-reduced chenodoxycholic acid, 3α,5β-testosterone, and deoxy-corticosterone.

Other examples of 11β-HSD1 dehydrogenase inhibitors include 3α,5α-TH-aldosterone, 3α,5α-TH-cortisol, 5α-DH-corticosterone, 3α,5α-TH-corticosterone, 3α,5α-TH-11-dehydro-corticosterone, 11β-OH-allopregnanolone, 11β-OH-pregnanolone, 11β-OH-androstanediol, and 3β,5α-reduced steroids.

In another embodiment, the 11β-HSD1 dehydrogenase inhibitor is a 3α,5α-reduced steroid. Examples of such steroids include 3α,5α-reduced-11β-OH-progesterone, 3α,5α-reduced-11β-OH-testosterone, 3α,5α-reduced-11β-OH-androstendione, 3α,5α-reduced-11β-OH-pregnenolone, 3α,5α-reduced-11β-OH-dehydro-epiandrostenedione, 3α,5α-reduced-corticosterone, 3α,5α-reduced-aldosterone, 3α,5α-reduced-pregnenolone, 3α,5α-reduced-progesterone, 3α,5α-reduced testosterone, 3α,5α-deoxycorticosterone, and 3α,5α-reduced-chenodeoxycholic acid. Other examples of steroids which can be used as 11β-HSD1 dehydrogenase inhibitors include 11β-OH progesterone, 11β-OH testosterone, 11β-OH-pregnenolone, 11β-OH-dehydro-epiandrostenedione, glycyrrhetinic acid or carbenoxolone.

In one embodiment, the 11β-HSD1 dehydrogenase inhibitor has an IC₅₀ of 0.5 μM or less. In another embodiment, the 11β-HSD1 dehydrogenase inhibitor has an IC₅₀ of 100 μM or less, 80 μM or less, or 20 μM or less (using 100 nM corticosterone substrate concentration and testicular Leydig cell homogenates).

The term “11β-HSD2 dehydrogenase inhibitor” includes agents which inhibit or decrease the dehydrogenase activity of 11β-HSD2.

In one embodiment, the 11β-HSD2 dehydrogenase inhibitor is a small molecule, such as a steroid or a derivative thereof. In one embodiment, the steroid is 3α,5α-reduced. Examples of 11β-HSD2 dehydrogenase inhibitors include, but are not limited to, 3α,5α-reduced-11β-OH-progesterone, 3α,5α-reduced-11β-OH-testosterone, 3α, 5α-reduced-11β-OH-androstenedione, 3α,5α-reduced-11-keto-progesterone, 3α, 5α-reduced-11-dehydro-corticosterone, 3α,5α-reduced-corticosterone, 3α,5α-reduced-11β-OH-pregnenolone, 3α, 5α-reduced-11β-OH-dehydro-epiandrostenedione, 3α,5α-reduced-pregnenolone, 3α,5α-reduced-dehydro-epiandrostenedione, 3α,5α-reduced aldosterone, and 3α, 5α-reduced deoxycorticosterone. Other examples of 11β-HSD2 dehydrogenase inhibitors include 11β-OH-progesterone, 11β-OH-pregnenolone, 11β-OH-dehydro-epiandrostenedione, 11β-OH-testosterone, 11-keto-progesterone, 5α-dihydro-corticosterone, 3α,5α-reduced deoxy-corticosterone, glycyrrhetinic acid or carbenoxolone.

Other examples of 11β-HSD2 dehydrogenase modulating (e.g., inhibiting compounds) include: 3β,5α-reduced steroids, 3α,5α-TH-aldosterone, 3α,5α-TH-cortisol, 5α-DH-corticosterone, 11-dehydro-corticosterone, 3α,5α-TH-11-dehydrocorticosterone, 11-keto-allopregnanolone, 11β-OH-androstanediol, 11β-OH-androstenedione, and pharmaceutically acceptable salts and prodrugs thereof.

In other embodiments, 11β-HSD2 dehydrogenase modulating compound is a nucleic acid. In another embodiment, the 11β-HSD2 dehydrogenase inhibitor is an antisense nucleic acid. In another embodiment, the 11β-HSD2 dehydrogenase inhibitor is a siRNA.

In one embodiment, the 11β-HSD2 dehydrogenase inhibiting compounds have IC₅₀'s less than 2.5 μM (using 50 nM corticosterone substrate concentration and sheep kidney microsomes). In another embodiment, the 11β-HSD2 dehydrogenase inactive compounds have an IC₅₀ of less than 10 μM.

Examples of 11β-HSD1-reductase, 11β-HSD1-dehydrogenase and 11β-HSD2 dehydrogenase modulating compounds are described in Table 1.

TABLE

| Compound | | 11β-HSD1 | 11β-HSD1 | 11β-HSD2 |
|---|---|---|---|---|
| Name | Structure | Reductase | Dehydrogenase | Dehydrogenase |
| 11β-OH- progesterone | 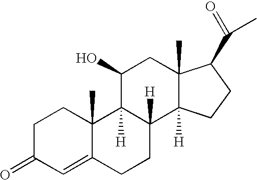 | No Inhibition | Potent Inhibitor (Non-Selective) | Potent Inhibitor (Non-Selective) |
| 11β-OH- testosterone | 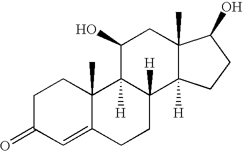 | No Inhibition | Inhibitor (Non-Selective) | Inhibitor (Non-Selective) |
| 3α,-5β-reduced- 11β-OH- testosterone | 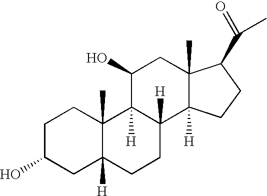 | No Inhibition | Moderate Inhibition | No Inhibition |
| 3α,-5β-reduced- 11β-OH- testosterone | 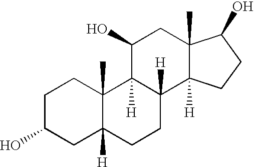 | No Inhibition | Moderate Inhibition | No Inhibition |
| chenodeoxycholic acid (3α,5β-reduced steroid) | 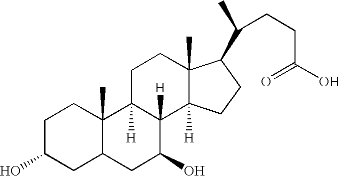 | No Inhibition | Selective inhibitor | No Inhibition |
| 3α,5α-reduced- 11β-OH- progesterone | 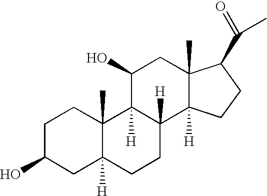 | No Inhibition | Potent Inhibitor (Non-Selective) | Potent Inhibitor (Non-Selective) |
| 3α,5α-reduced- 11β-OH- testosterone | 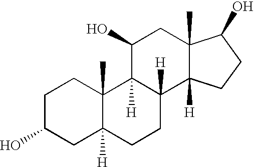 | No Inhibition | Potent Inhibitor (Non-Selective) | Potent Inhibitor (Non-Selective) |
| 3α,5α-reduced- 11β-OH- androstenedione | 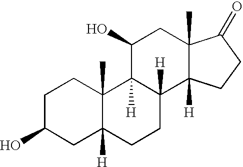 | No Inhibition | Moderate Inhibitor | Potent Inhibitor (Non-Selective) |
| 11-Keto- progesterone | 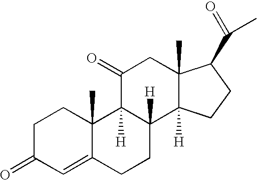 | Selective Inhibitor | No Inhibition | Potent Inhibitor |
| 11-Keto- testosterone | 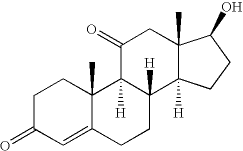 | Selective Inhibitor | No Inhibition | No Inhibition |
| 11-Keto- androstenedione | 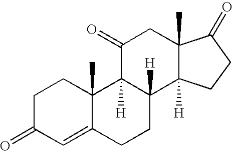 | Selective Inhibitor | No Inhibition | No Inhibition |
| 3α,5α-reduced- 11-keto- progesterone | 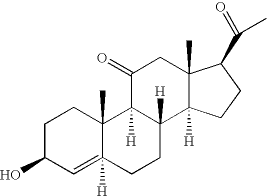 | Selective Inhibitor | No Inhibition | Potent Inhibitor |
| 3α,5α-reduced- 11-keto- testosterone | 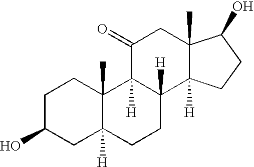 | Selective Inhibitor | No Inhibition | Not tested |
| 3α,5α-reduced- 11-keto- androstenedione | 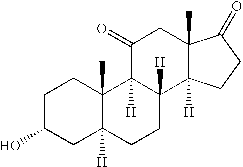 | Selective Inhibitor | No Inhibition | Not tested |
| 3α,5α-tetrahydro- 11-dehydro- corticosterone | 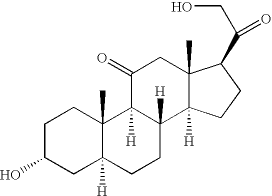 | Potent Inhibitor | No Inhibition | Potent Inhibitor |
| 3α,5α-reduced- corticosterone | 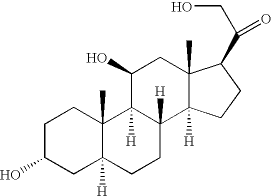 | No Inhibition | Potent Inhibitor | Potent Inhibitor |
| 5α-dihydro- corticosterone | 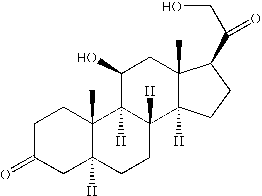 | No inhibition | Potent Inhibitor | Potent Inhibitor |
| 3α,5α-reduced aldosterone | 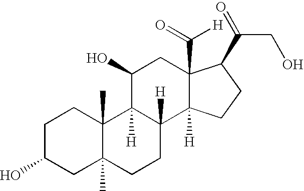 | No Inhibition | Moderate Inhibitor | Potent Inhibitor |

IV. Pharmaceutical Compositions

In yet another embodiment, the invention pertains to a pharmaceutical composition for increasing or decreasing male fertility. The composition includes an effective amount of an 11β-HSD1 reductase, 11β-HSD1 dehydrogenase, or 11β-HSD2 dehydrogenase modulating, e.g., inhibiting, compound and a pharmaceutically acceptable carrier. In another embodiment, the pharmaceutical compositions may also comprise an inhibitor of 17α-hydroxylase, 20α-reductase or 20β-reductase. In another embodiment, the invention also features a pharmaceutical composition comprising an effective amount of a 11β-HSD1 reductase, 11β-HSD1 dehydrogenase, or 11β-HSD2 dehydrogenase modulating, e.g., inhibiting, compound, for modulating testosterone levels in a subject.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal, pulmonary and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. Sprays also can be delivered by mechanical, electrical, or by other methods known in the art.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial, antiparasitic and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form may be accomplished by dissolving or suspending the drug in an oil vehicle. The compositions also may be formulated such that its elimination is retarded by methods known in the art.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration or administration via inhalation is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually. Other methods for administration include via inhalation.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous and subcutaneous doses of the compounds of this invention for a patient will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats a glucocorticoid associated state.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

As set out above, certain embodiments of the present compounds can contain a basic functional group, such as amino or alkylamino, and are, thus, capable of forming pharmaceutically acceptable salts with pharmaceutically acceptable acids. The term “pharmaceutically acceptable salts” is art recognized and includes relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Farm. SCI. 66:1-19).

In other cases, the compounds of the present invention may contain one or more acidic functional groups and, thus, are capable of forming pharmaceutically acceptable salts with pharmaceutically acceptable bases. The term “pharmaceutically acceptable salts” in these instances includes relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. Hydroxyls can be converted into esters via treatment with an esterifying agent such as alkanoyl halides. The term also includes lower hydrocarbon groups capable of being solvated under physiological conditions, e.g., alkyl esters, methyl, ethyl and propyl esters. (See, for example, Berge et al., supra.)

The invention also pertains to any one of the methods described supra further comprising administering to the subject a pharmaceutically acceptable carrier.

### EXEMPLIFICATION OF THE INVENTION

### Example 1

### Ability of Corticosterone and 11-Dehydro-Corticosterone to Amplify the Contractile Responses of Phenylephrine

Experimental:

Male Sprague-Dawley (150-200 g) rats were anesthetized with pentobarbital (50 mg/kg IP), and a median sternotomy was performed followed by the rapid removal of the thoracic aorta. The adventitia was removed, but the endothelium was left intact. The aorta was cut into 2-3 mm rings and individual rings were placed into a single well of a twenty four well culture plate and incubated at 37° C. under 95% O₂-5% CO₂. Each well contained 1 mL of DMEM/F12 containing 1% fetal bovine serum, streptomycin (100 μg/ml), penicillin (100 units/ml) and amphotericin (0.25 μg/ml). Aortic rings were incubated for 24 hours prior to contractility measurements with the following combinations of steroids, and antisense/nonsense oligonucleotides (3 μmol/L):

Corticosterone (10 nmol/L)+11β-HSD2 antisense or 11β-HSD2 nonsense oligomer

Corticosterone (10 nmol/L)+11β-HSD1 antisense or 11β-HSD1 nonsense oligomer

In 11-dehydrocorticosterone experiments with vehicle alone

11-dehydrocorticosterone (100 nmol/L)+11β-HSD1 Antisense or 11β-HSD1 nonsense oligomer

Antisense phosphorothioate oligonucleotides, targeted to block either 11β-HSD2 or 11β-HSD1 gene expression, were obtained from Research Genetics, Huntsville Ala. Antisense oligomers complementary to 20 bp sequences spanning the ribosome binding/translation start site were used. Oligomer sequences were: 5′-CAT AAC TGC CGT CCA ACA GC-3′ (SEQ ID No.: 2) for 11β-HSD1 Antisense and 5′-AGC CCA GCG CTC CAT GAC TT-3′ (SEQ ID No. 3) for 111-HSD2 antisense. In control experiments the corresponding sense sequence was used as the nonsense oligomer. Antisense and nonsense oligomers were added directly to each well at 20 μg/10:1 sterile H₂0 per well for a final concentration of 3 μmol/L.

For contraction measurements, aortic rings were suspended by tungsten wires with 1 g of tension and placed in a vessel bath containing serum free DMEM/F12 media at 37° C. aerated with 95% O₂-5% CO, at pH 7.4. Vessels were equilibrated for 20 minutes and then tested with phenylephrine (1 mol/L-10 μmol/L). Although phenylephrine is structurally not a catecholamine, it is considered to be a functional catecholaamine as it activates both α and β adrenoceptors. Due to its favorable stability characteristics, it is widely used as a catecholamine substitute in experiments of this nature. The intensity of contraction was assessed by use of a Narishige micromanipulator and model FT03 force transducer (Grass Instrument Co. West Warwick, R.I.). Measurements were recorded on computer using the Labview 4.1 Virtual Instrument System (National Instruments, Austin, Tex.). Adhering to this protocol, test vessel viability by demonstrating the ability of the vessel to vigorously contract when exposed to known vasoconstrictors and relax back to baseline after treatment with acetylcholine.

Results: Effect of 110-HSD Antisense on Vascular Contractile Response

Experiments were carried out to determine whether specific 11β-HSD2 antisense oligomers affect the contractile response of vascular rings. Rat aortic rings, with endothelium intact, were incubated for 24 hours with corticosterone (10 nmol/L) and either specific 11β-HSD2 antisense oligomers (3 μmol/L) or nonsense oligomers (3 (μmol/L). Following incubation, the contractile responses to graded concentrations of phenylephrine were determined. Previously, it had been demonstrated that the incubation of aortic rings with corticosterone resulted in amplified contractile responses to graded concentrations of phenylephrine compared to controls. The exposure of rings to corticosterone together with 11β-HSD2 antisense demonstrated a statistically significant increase in the contractile response to all concentrations (1, 10, 100 nmol/L and 1 μmol/L) of phenylephrine.

In the rat, both vascular endothelial and smooth muscle cells contain 11β-HSD1. Even though this isoform operates mainly as a reductase under physiologic conditions, it was examined if 11β-HSD1 antisense oligomers had an effect on the ability of corticosterone to amplify the contractile responses to phenylephrine in vascular tissue. Rings were incubated for 24-hours with corticosterone (10 nmol/L) and either 11β-HSD1 antisense oligomers (3 μmol/L) or nonsense oligomers (3 μmol/L). In rings treated with 11β-HSD1 antisense the contractile responses to all concentrations of phenylephrine (10 nmol/L, 100 nmol/L and 1 μmol/L) were significantly increased compared to rings treated with corticosterone and nonsense oligomers.

In rat vascular tissue, 11β-HSD1 acts predominantly as a reductase metabolizing inactive 11-dehydro-glucocorticoid back to the active parent hormone. 11-dehydro-corticosterone ((just like corticosterone) also amplifies the contractile responses to phenylephrine in rat aortic rings. In the rat, 11β-HSD1 is present in both vascular endothelial and smooth muscle cells and under physiological conditions this enzyme functions predominantly as a reductase.

Furthermore, the effect of 11β-HSD1 antisense oligomers on the ability of 11-dehydro-corticosterone to amplify the contractile responses to phenylephrine was studied. Rings were incubated for 24 hours with 11-dehydro-corticosterone (100 nmol/L) and either 11β-HSD1 antisense (3 μmol/L) or nonsense (3 μmol/L) oligomers. 11β-HSD1 antisense oligomers attenuated the ability of 11β-dehydro-corticosterone to amplify the contractile response to all concentrations of phenylephrine compared to 11-dehydro-corticosterone plus 11β-HSD1 nonsense oligomers. Statistically significant decreases were observed at 100 nmol/L and 1 μmol/L phenylephrine.

In aortic rings incubated (24-hours) with corticosterone (10 nmol/L) and 11β-HSD2 antisense (3 μmol/L), the contractile response to graded concentrations of phenylephrine (PE: 10 nmol/L-1 μmol/L) were significantly (P<0.05) increased compared to rings incubated with corticosterone and 11β-HSD2 nonsense. 11β-HSD1 antisense oligomers also enhanced the ability of corticosterone to amplify the contractile response to phenylephrine.

Discussion

Earlier experiments showed that inhibitors of 11β-HSD dehydrogenase activity enhance the ability of corticosterone to amplify the vasoconstrictive actions of phenylephrine and angiotensin II in rat aorta. The examples show that a specific 11β-HSD2 antisense oligomer also enhances the ability of corticosterone to amplify the contractile responses of catecholamines. Since 11β-HSD2 appears to exist only in endothelial cells, this observation supports a role for the action of glucocorticoids in affecting endothelial cell function. Although 11β-HSD1 acts predominantly as a reductase in vascular tissue, 11β-HSD1 antisense oligomers also enhanced the ability of corticosterone to amplify the contractile effects of phenylephrine in rat aortic rings. This observation suggests that 11β-HSD1-dehydrogenase, in addition to 11β-HSD2, also operates to protect GR and MR from over-activation by glucocorticoids in vascular tissue. Further experiments to determine whether antisense oligomers down-regulate mRNA and protein expression of their respective 11β-HSD1 isoform under conditions in which they enhance contractile responses in aortic rings will be done. Using a similar protocol to the one described here, it has been shown using RT-PCR analysis, that 11β-HSD2 antisense and 11β-HSD1 antisense down-regulate the expression of their respective enzyme isoforms in cultured rat vascular endothelial and smooth muscle cells.

The example confirms that 11-dehydro-corticosterone also amplifies the contractile actions of catecholamines in rat aortic rings. Since 11-dehydro-glucocorticoids do not bind to GR (or MR) to any major extent, it is proposed that 11-dehydro-corticosterone is metabolized back to corticosterone by 11β-HSD1-reductase in vascular smooth muscle and/or endothelial cells. This hypothesis is supported by the discovery that 11-keto-progesterone, a specific inhibitor of 11β-HSD1-reductase activity (backward reaction), diminished the ability of 11-dehydro-corticosterone to amplify the contractile effects of phenylephrine and decreased the metabolism of 11-dehydro-corticosterone back to corticosterone. The examples also demonstrate that 11β-HSD1 antisense oligomer also attenuates the ability of 11-dehydro-corticosterone to amplify the contractile responses of phenylephrine indicating that the down-regulation of 11β-HSD1 gene expression can affect the regeneration of active glucocorticoid (from 11-dehydro-glucocorticoid) in vascular tissue. Indeed, the examples show that 11β-HSD1 antisense can significantly reduce the metabolism of 11-dehydro-corticosterone back to corticosterone in aortic ring preparations.

### Example 2

### Metabolism of Corticosterone and 11-Dehydro-Corticosterone in Vascular Tissue

Experimental:

The effects of 11β-HSD1 and 11β-HSD2 antisense on the inter-conversion of ³H-corticosterone and ³H-11-dehydro-corticosterone by aortic rings was also determined. Rings (2-3 mm) obtained in a similar manner as those in the contraction studies, were incubated in 1 ml DMEM/F12 media containing 1% FBS at 37° C. under 95% O₂-5% CO₂ in 24-well culture plates. Rings were incubated for 24 hours with:

(i) ³H-corticosterone (10 nmol/L)±11β-HSD2 or 11β-HSD1 antisense (3 μmol/L); control groups received nonsense oligomers. The amount of ³H-11-dehydro-corticqsterone in the incubation medium after 24 hrs was then measured. The effects of 11β-HSD1 antisense/nonsense were measured in quadruplicate (n=6 aortic rings per well) and the effects of 11β-HSD2 antisense/nonsense in duplicate (n=8 aortic rings per well),

(ii) ³H-11-dehydro-corticosterone (10 nmol/L)±11β-HSD1 antisense (3 μmol/L); this experiment was performed in duplicate (n=10 aortic rings per well). Control groups were incubated with the appropriate nonsense oligomer. ³H-corticosterone in the incubation medium after 24 hrs was then measured. In this experiment, aortic rings were also analyzed for ³H-corticosterone content. Rings from duplicate incubations (total n=20) were blotted dry, pooled and homogenized in 50% methanol using a Polytron. The homogenates were then centrifuged, extracted as below using Sep-Paks and injected onto a HPLC system for analysis.

Incubation media was collected, ran through a Sep-Pak and eluted with 3 mls of methanol, the eluate was then dried under nitrogen and reconstituted in 500:1 methanol. The aortic rings were dried and weighed. The steroids present in the eluate were separated by high-pressure liquid chromatography with a Dupont Zorbax C8 column eluted at 44° C. at a flow rate of 1 mL/min using 55% methanol for 10 minutes. Steroids were observed by monitoring radioactivity on-line with a Packard Radiomatic Flo-One/Beta Series A-500 counter connected to a Dell Optiflex 425 S/L computer. Corticosterone and 11-dehydro-corticosterone were identified by comparing their retention times with that of known standards.

Corticosterone and phenylephrine were obtained from Sigma (St Louis, Mo.), 11-dehydrocorticosterone from Research Plus (Bayonne, N.J.) and ³H-steroids from New England Nuclear (Boston, Mass.). Where appropriate, data were expressed as mean±SE and analyzed using ANOVA and the Student's t test with Bonferroni modification. P values of less than 0.05 are considered significant.

Results: Effects of 11β-HSD Antisense on Steroid Metabolism

A series of experiments were then conducted to test whether 11β-HSD2 and 11β-HSD1 antisense oligomers did affect the enzymatic conversion of corticosterone and 11-dehydrocorticosterone. In experiments in which aortae were taken from rats (n=4) and 6 rings cut from each aorta were incubated for 24 hrs with ³H-corticosterone (10 nM) plus 11β-HSD1 antisense (3 μM), the conversion of corticosterone to 11-dehydrocorticosterone was 21% lower than in aortic rings incubated with corticosterone and 11β-HSD1 nonsense oligomers. In a further two experiments, aortae were taken from rats (n=2) and 8 aortic rings cut from each. Aortic ring preparations incubated for 24 hrs with corticosterone and 11β-HSD2 antisense (3 μM), demonstrated a 24% reduction in the conversion of corticosterone to 11-dehydrocorticosterone compared to aortic rings incubated with corticosterone and 11β-HSD2 nonsense.

To determine the effects of 11β-HSD1 antisense on 11β-HSD1-reductase activity rat aortae were taken from rats (n=2) and 10 aortic rings cut from each. These aortic rings were then incubated for 24 hours with ³H-11-dehydrocorticosterone and either 11β-HSD1 antisense or nonsense and the production of corticosterone was measured. The production of ³H-corticosterone was markedly reduced in rings incubated with 11βHSD1 antisense compared to rings incubated with 11β-HSD1 nonsense oligomers. Thus, 11β-HSD1 antisense profoundly diminished the ability of the rat aortic rings to metabolize 11-dehydro-corticosterone back to corticosterone. The aortic ring tissue in these experiments was also pooled (n=20) and analyzed for steroid content. The amount of radioactivity in the tissue was approximately 2-3% of the total radioactivity in the incubation media. The production of ³H-corticosterone in aortic rings incubated with 11β-HSD1 antisense was again markedly lower that that in rings incubated with 11β-HSD1 nonsense oligomers. The levels of ³H-11-dehydrocorticosterone metabolism measured in the incubate and in the aortic tissue were very similar. This indicates that measuring steroid content in the media does not under-represent the level of steroid metabolism in the tissue compartment.

Discussion

In this example, experiments were undertaken to determine whether antisense oligomers could affect 11β-HSD enzyme activity and, indeed, it has been demonstrated that 11β-HSD2 and 11β-HSD1 antisense caused moderate reductions (24 and 21% respectively) in the metabolism of corticosterone. These reductions in metabolism translate to relatively small increases in residual corticosterone levels in the aortic ring tissue that would not appear to account for the relatively large increases in phenylephrine-induced vasoconstriction observed in the contractile studies. However, glucocorticoids have been reported to not only amplify the contractile effects of catecholamines in vascular tissue but to also diminish the effects of certain vasorelaxation pathways (glucocorticoids decrease nitric oxide and prostaglandin I₂ synthesis); such actions would serve to further enhance the effects of glucocorticoids on increasing catecholamine-induced vasoconstriction and may explain how small changes in glucocorticoid levels can have profound effects on vascular tone.

In addition, 11β-HSD2 and 11β-HSD1 antisense also decreased the metabolism of corticosterone to 11-dehydro-corticosterone. 11-dehydro-corticosterone (100 nmol/L) also amplified the contractile response to phenylephrine in aortic rings (P<0.01), most likely due to the generation of active corticosterone by 11β-HSD1-reductase; this effect was significantly attenuated by 11β-HSD1 antisense. 11β-HSD1 antisense also caused a marked decrease in the metabolism of 11-dehydro-corticosterone back to corticosterone by 11β-HSD1-reductase. These findings underscore the importance of 11β-HSD2 and 11β-HSD1 in regulating local concentrations of glucocorticoids in vascular tissue. They also indicate that decreased 11β-HSD2 activity may be a possible mechanism in hypertension and other blood pressure associated disorders and that 11β-HSD1-reductase may be a possible target for anti-hypertensive therapy.

The results of these examples underscore the importance of 11β-HSD2 in regulating the access of glucocorticoids to GR and/or MR in vascular tissue and suggest that 11β-HSD1-dehydrogenase may also play a role in protecting GR and MR in this tissue. In addition, they suggest that the antisense oligomers used in these experiments down-regulate 11β-HSD gene expression and decrease glucocorticoid metabolism in vascular tissue, an effect leading to increased vascular responsiveness to catecholamines.

The examples also demonstrate that both 11β-HSD2 and 11β-HSD1 regulate local glucocorticoid concentrations in vascular tissue with 11β-HSD2 and 11β-HSD1-dehydrogenase working to decrease- and 11β-HSD1-reductase increase the amount of glucocorticoid that can access GR and MR in vascular smooth muscle. Physiological concentrations of both free corticosterone and 11-dehydrocorticosterone are similar over the course of the day in rodents. Therefore significant quantities of not only glucocorticoid, but also of 11-dehydro-glucocorticoid are available for conversion back to the glucocorticoid. Since glucocorticoids amplify catecholamine and angiotensin II pressor responses and may inhibit the effects of some vasorelaxant pathways, a possible mechanism that may increase vascular tone and induce hypertension includes a decrease in 11β-HSD2 activity. Interestingly, many patients with essential hypertension also demonstrate decreased 11β-HSD2 activity as assessed by altered plasma and urinary cortisolxortisone ratios. Moreover, the plasma half-life of 11α-³H-cortisol is prolonged in patients with essential hypertension consistent with the idea that 11β-HSD2 activity is diminished in this condition. The present work also suggests that since 11β-HSD1 reductase generates active glucocorticoid in vascular tissue, a possible therapeutic target in the treatment of hypertension could be the specific inhibition of 11β-HSD1 reductase activity.

### Example 3

### Effects of Steroids on Rat Leydig Cells NADPH Production

Whole cell preparations of rat Leydig cells were incubated with 11β-hydroxy-3α5α TH progesterone, 3α5α TH-testosterone, and 3α5α TH-corticosterone. A three to five fold increase in NADPH production was noted in each experiment. These experiments proved that 3α,5α-corticosterone stimulates 11β-HSD1 dehydrogenase, and production of NADPH which can be used for testosterone biosynthesis.

Similar experiments were performed with 3α,5β-deoxycorticosterone and 3α,5β progesterone, neither of which posess an 11β-hydroxy group. Neither of these compounds effected the backward reaction of 11β-HSD1 reductase.

Equivalents

Those skilled in the art will recognize, or be able to ascertain using no more than routine experimentation, many equivalents to the specific embodiments and methods described herein. Such equivalents are intended to be encompassed by the scope of the following claims.

All patents, patent applications, and literature references cited herein are hereby expressly incorporated by reference. This application is related to U.S. Ser. No. 10/835,890, the entire contents of which are hereby incorporated herein.

1. A method for increasing male fertility, comprising administering an effective amount of a 11β-HSD1 reductase inhibitor to a subject, such that said fertility is increased, wherein said 11β-HSD1 reductase inhibitor is a non-competitive inhibitor.

2. A method for increasing testosterone levels in a subject, comprising administering to said subject an effective amount of a 11β-HSD1 reductase inhibitor, such that testosterone levels in said subject are increased, wherein said 11β-HSD1 reductase inhibitor is a non-competitive inhibitor.

3. The method of claim 1 or 2, wherein said 11β-HSD1 reductase inhibitor is selective for testicular 11β-HSD1 reductase.

4. The method of claim 1 or 2, wherein said 11β-HSD1 reductase inhibitor is a steroid derivative.

5. A method for increasing male fertility, comprising administering an effective amount of a 11β-HSD1 reductase inhibitor to a subject, such that said fertility is increased, wherein said 11β-HSD1 reductase inhibitor is 3β,5α-reduced steroid, 3α,5α-TH-cortisone, 3α,5β-TH-cortisone, 5α-DH-corticosterone, 3α,5α-TH-corticosterone, 3α,5α-TH-11-dehydro-corticosterone, 11β-OH-pregnanolone, 11-keto-allopregnanolone, 11-keto-androstenedione, and pharmaceutically acceptable prodrug or salts thereof.

6. A method for increasing testosterone levels in a subject, comprising administering to said subject an effective amount of a 11β-HSD1 reductase inhibitor, such that testosterone levels in said subject are increased, wherein said 11β-HSD1 reductase inhibitor is a 3β, 5αe-reduced steroid, 3α,5α-TH-cortisone, 3α,5α-TH-cortisone, 5α-DH-corticosterone, 3α,5α-TH-corticosterone, 3α,5α-TH-11-dehydro-corticosterone, 11β-OH-pregnanolone, 11-keto-allopregnanolone, 11-keto-androstenedione, and pharmaceutically acceptable prodrug or salts thereof.

7. A method for decreasing male fertility, comprising administering to a subject an effective amount of a 11β-HSD1 dehydrogenase inhibitor, such that said fertility is decreased, wherein said inhibitor is a non-competitive inhibitor.

8. A method for decreasing testosterone levels in a subject, comprising administering to a subject an effective amount of a 11β-HSD1 dehydrogenase inhibitor, such that testosterone levels in said subject are decreased, wherein said inhibitor is non-competitive.

9. A method for decreasing male fertility, comprising administering to a subject an effective amount of a 11β-HSD1 dehydrogenase inhibitor, such that said fertility is decreased, wherein said inhibitor is a 3β,5α-reduced steroid, 3α,5α-TH-aldosterone, 3α,5α-TH-cortisol, 5α-DH-corticosterone, 3α,5α-TH-corticosterone, 3α,5α-TH-11-dehydro-corticosterone, 11β-OH-allopregnanolone, 11β-OH-pregnanolone, 11β-OH-androstanediol, or a pharmaceutically acceptable prodrug or salt thereof.

10. A method for decreasing testosterone levels in a subject, comprising administering to a subject an effective amount of a 11β-HSD1 dehydrogenase inhibitor, such that testosterone levels in said subject are decreased, wherein said inhibitor is 3β, 5α-reduced steroid, 3α,5α-TH-aldosterone, 3α,5α-TH-cortisol, 5α-DH-corticosterone, 3α,5α-TH-corticosterone, 3α,5α-TH-11-dehydro-corticosterone, 11β-OH-allopregnanolone, 11β-OH-pregnanolone, 11β-OH-androstanediol, or a pharmaceutically acceptable prodrug or salt thereof.

11. A method of increasing testosterone biosynthesis, comprising administering to a male subject an effective amount of a 11β-OH steroid or 11-keto steroid, such that testosterone biosynthesis is increased.

12. A method of decreasing testosterone biosyntheis, comprising administering to a male subject an effective amount of an 11-deoxy steroid, such that testosterone levels are decreased.

13. The method of claim 12, wherein said 11-deoxy steroid is 3α,5β-tetrahydrodeoxycorticosterone, 3α,5β-tetrahydroprogesterone, or chenodeoxycholic acid.

14. The method of claim 12, wherein said effective amount is effective to decrease fertility, or to treat prostate cancer or prostate disease.

15. A method of treating a hypergonadism associated disorder, comprising administering to said subject an effective amount of a 11β-HSD1 reductase inhibitor, such that said hypergonadism associated disorder is treated.

16. The method of claim 15, wherein said hypergonadism associated disorder is obesity, insulin resistance, or metabolic syndrome.

17. The method of claim 15, wherein said 11β-HSD1 reductase inhibitor is a non-competitive inhibitor.

18. The method of claim 15, wherein said 11β-HSD1 reductase inhibitor is a 11-keto steroid.
